SEQUENCE LISTING (28 sequences)

SEQ ID NO: 1 - protein, 9 aa, synthetic construct
  source 1..9: note = Degron Wildtype
FYTVWRAFL

SEQ ID NO: 2 - protein, 8 aa, synthetic construct
  source 1..8: note = Degron Mutant_No F
YTVWRAFL

SEQ ID NO: 3 - protein, 8 aa, synthetic construct
  source 1..8: note = Degron Mutant_ No L
FYTVWRAF

SEQ ID NO: 4 - protein, 9 aa, synthetic construct
  source 1..9: note = Degron Mutant_W-A
FYTVARAFL

SEQ ID NO: 5 - protein, 7 aa, synthetic construct
  source 1..7: note = Degron Mutant_ No F_No L
YTVWRAF

SEQ ID NO: 6 - protein, 8 aa, synthetic construct
  source 1..8: note = Degron Mutant_No F_W-A
YTVARAFL

SEQ ID NO: 7 - protein, 8 aa, synthetic construct
  source 1..8: note = Degron Mutant_No L_W-A
FYTVARAF

SEQ ID NO: 8 - protein, 7 aa, synthetic construct
  source 1..7: note = Degron Mutant_No F_No L_W-A
YTVARAF

SEQ ID NO: 9 - DNA, 27 bp, synthetic construct
  source 1..27: mol_type = other DNA; note = Nucleotide Sequence of Degron Wildtype
ttctatacagtgtggcgcgctttttta

SEQ ID NO: 10 - DNA, 24 bp, synthetic construct
  source 1..24: mol_type = other DNA; note = Nucleotide Sequence of Degron Mutant_ No F
tatacagtgtggcgcgctttttta

SEQ ID NO: 11 - DNA, 24 bp, synthetic construct
  source 1..24: mol_type = other DNA; note = Nucleotide Sequence of Degron Mutant_No L
ttctatacagtgtggcgcgctttt

SEQ ID NO: 12 - DNA, 27 bp, synthetic construct
  source 1..27: mol_type = other DNA; note = Nucleotide Sequence of Degron Mutant_ W-A
ttctatacagtggcgcgcgctttttta

SEQ ID NO: 13 - DNA, 21 bp, synthetic construct
  source 1..21: mol_type = other DNA; note = Nucleotide Sequence of Degron Mutant_No F_No L
tatacagtgtggcgcgctttt

SEQ ID NO: 14 - DNA, 24 bp, synthetic construct
  source 1..24: mol_type = other DNA; note = Nucleotide Sequence of Degron Mutant_No F_W-A
tatacagtggcgcgcgctttttta

SEQ ID NO: 15 - DNA, 24 bp, synthetic construct
  source 1..24: mol_type = other DNA; note = Nucleotide Sequence of Degron Mutant_No L_W-A
ttctatacagtggcgcgcgctttt

SEQ ID NO: 16 - DNA, 21 bp, synthetic construct
  source 1..21: mol_type = other DNA; note = Nucleotide Sequence of Degron Mutant_No F_No L_W-A
tatacagtggcgcgcgctttt

SEQ ID NO: 17 - protein, 250 aa, synthetic construct
  source 1..250: note = GFP Degron Fusion Protein
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSFYTVWRAFL

SEQ ID NO: 18 - protein, 360 aa, synthetic construct
  source 1..360: note = MTCH Degron Fusion Protein
MDYKDDDDKYPYDVPDYASGRMADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLISRCGAGTVTFLSSQMHRIMGENVDFYTVWRAFLIII

SEQ ID NO: 19 - protein, 349 aa, synthetic construct
  source 1..349: note = Renilla luciferase Degron Fusion Protein
MTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQSGSRCGAGTVTFLSSQMHRIMGENVDFYTVWRAFLIII

SEQ ID NO: 20 - protein, 302 aa, synthetic construct
  source 1..302: note = mtDsRed Degron Fusion Protein
MSVLTPLLLRGLTGSARRLPVPRAKIHSLGDPPVATMASSENVITEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVATVTQDSSLQDGCFIYKVKFIGVNFPSDGPVMQKKTMGWEASTERLYPRDGVLKGETHKALKLKDGGHYLVEFKSIYMAKKPVQLPGYYYVDAKLDITSHNEDYTIVEQYERTEGRHHLFLGTELGSRCGAGTVTFLSSQMHRIMGENVDFYTVWRAFLIII

SEQ ID NO: 21 - DNA, 753 bp, synthetic construct
  source 1..753: mol_type = other DNA; note = Nucleotide Sequence of GFP Degron Fusion Protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatccttctatacagtgtggcgcgcttttttataa

SEQ ID NO: 22 - DNA, 1083 bp, synthetic construct
  source 1..1083: mol_type = other DNA; note = Nucleotide Sequence of MTCH Degron Fusion Protein
atggactacaaggacgacgatgacaagtacccttatgacgtgcccgattacgctagcggccgcatggcggacgcggccagtcaggtgctcctgggctccggtctcaccatcctgtcccagccgctcatgtacgtgaaagtgctcatccaggtgggatatgagcctcttcctccaacaataggacgaaatatttttgggcggcaagtgtgtcagcttcctggtctctttagttatgctcagcacattgccagtatcgatgggaggcgcgggttgttcacaggcttaactccaagactgtgttcgggagtccttggaactgtggtccatggtaaagttttacagcattaccaggagagtgacaagggtgaggagttaggacctggaaatgtacagaaagaagtctcatcttcctttgaccacgttatcaaggagacaactcgagagatgatcgctcgttctgctgctaccctcatcacacatcccttccatgtgatcactctgagatctatggtacagttcattggcagagaatccaagtactgtggactttgtgattccataataaccatctatcgggaagagggcattctaggatttttcgcgggtcttgttcctcgccttctaggtgacatcctttctttgtggctgtgtaactcactggcctacctcgtcaatacctatgcactggacagtggggtttctaccatgaatgaaatgaagagttattctcaagctgtcacaggattttttgcgagtatgttgacctatccctttgtgcttgtctccaatcttatggctgtcaacaactgtggtcttgctggtggatgccctccttactccccaatatatacgtcttggatagactgttggtgcatgctacaaaaagaggggaatatgagccgaggaaatagcttatttttccggaaggtcccctttgggaagacttattgttgtgacctgaaaatgttaatttcaagatgtggggcagggacagtgacatttctgtcgtcccagatgcacagaattatgggagagaatgttgatttctatacagtgtggcgcgcttttttaataatcatttaa

SEQ ID NO: 23 - DNA, 1050 bp, synthetic construct
  source 1..1050: mol_type = other DNA; note = Nucleotide Sequence of Renilla luciferase Degron Fusion Protein
atgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaatcaggatccagatgtggggcagggacagtgacatttctgtcgtcccagatgcacagaattatgggagagaatgttgatttctatacagtgtggcgcgcttttttaataatcatttaa

SEQ ID NO: 24 - DNA, 909 bp, synthetic construct
  source 1..909: mol_type = other DNA; note = Nucleotide Sequence of mtDsRed Degron Fusion Protein
atgtccgtcctgacgccgctgctgctgcggggcttgacaggctcggcccggcggctcccagtgccgcgcgccaagatccattcgttgggggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgggtaccgagctcggatccagatgtggggcagggacagtgacatttctgtcgtcccagatgcacagaattatgggagagaatgttgatttctatacagtgtggcgcgcttttttaataatcatttaa

SEQ ID NO: 25 - DNA, 6189 bp, synthetic construct
  source 1..6189: mol_type = other DNA; note = Nucleotide Construct of GFP Degron Fusion Protein
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatccttctatacagtgtggcgcgcttttttataagcggccgctcgagtctagagggcccgcggttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 26 - DNA, 6330 bp, synthetic construct
  source 1..6330: mol_type = other DNA; note = Nucleotide Construct of MTCH Degron Fusion Protein
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcgatatcgccgccgccatggactacaaggacgacgatgacaagtacccttatgacgtgcccgattacgctagcggccgcatggcggacgcggccagtcaggtgctcctgggctccggtctcaccatcctgtcccagccgctcatgtacgtgaaagtgctcatccaggtgggatatgagcctcttcctccaacaataggacgaaatatttttgggcggcaagtgtgtcagcttcctggtctctttagttatgctcagcacattgccagtatcgatgggaggcgcgggttgttcacaggcttaactccaagactgtgttcgggagtccttggaactgtggtccatggtaaagttttacagcattaccaggagagtgacaagggtgaggagttaggacctggaaatgtacagaaagaagtctcatcttcctttgaccacgttatcaaggagacaactcgagagatgatcgctcgttctgctgctaccctcatcacacatcccttccatgtgatcactctgagatctatggtacagttcattggcagagaatccaagtactgtggactttgtgattccataataaccatctatcgggaagagggcattctaggatttttcgcgggtcttgttcctcgccttctaggtgacatcctttctttgtggctgtgtaactcactggcctacctcgtcaatacctatgcactggacagtggggtttctaccatgaatgaaatgaagagttattctcaagctgtcacaggattttttgcgagtatgttgacctatccctttgtgcttgtctccaatcttatggctgtcaacaactgtggtcttgctggtggatgccctccttactccccaatatatacgtcttggatagactgttggtgcatgctacaaaaagaggggaatatgagccgaggaaatagcttatttttccggaaggtcccctttgggaagacttattgttgtgacctgaaaatgttaatttcaagatgtggggcagggacagtgacatttctgtcgtcccagatgcacagaattatgggagagaatgttgatttctatacagtgtggcgcgcttttttaataatcatttaagaattcagtggatccactagtaacggccgccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggataattcctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagtgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 27 - DNA, 6486 bp, synthetic construct
  source 1..6486: mol_type = other DNA; note = Nucleotide Construct of Renilla luciferase Degron Fusion Protein
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaatcaggatccagatgtggggcagggacagtgacatttctgtcgtcccagatgcacagaattatgggagagaatgttgatttctatacagtgtggcgcgcttttttaataatcatttaagcggccgctcgagtctagagggcccgcggttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 28 - DNA, 6345 bp, synthetic construct
  source 1..6345: mol_type = other DNA; note = Nucleotide Construct of mtDsRed Degron Fusion Protein
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttatgtccgtcctgacgccgctgctgctgcggggcttgacaggctcggcccggcggctcccagtgccgcgcgccaagatccattcgttgggggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgggtaccgagctcggatccagatgtggggcagggacagtgacatttctgtcgtcccagatgcacagaattatgggagagaatgttgatttctatacagtgtggcgcgcttttttaataatcatttaagcggccgctcgagtctagagggcccgcggttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2025/0333748 A1
(43) Pub. Date: Oct. 30, 2025

(54) A DEGRON FOR REGULATING PROTEIN EXPRESSION AND METHODS THEREOF

(71) Applicant: INDIAN INSTITUTE OF SCIENCE, Bengaluru (IN)

(72) Inventors: Sandeep M. ESWARAPPA, Bengaluru (IN); Lekha E. MANJUNATH, Bengaluru (IN)

(21) Appl. No.: 19/074,999

(22) Filed: Mar. 10, 2025

(30) Foreign Application Priority Data
Feb. 23, 2024 (IN) 202441013294

(51) Int. Cl. C12N 15/67; C07K 7/06; C12N 9/02; C12N 15/62; C12N 15/85; G01N 33/68

(52) U.S. Cl. CPC C12N 15/67; C07K 7/06; C12N 9/0069; C12N 15/62; C12N 15/85; G01N 33/68; C07K 2319/42; C07K 2319/43; C07K 2319/60; C07K 2319/61; C07K 2319/70; C12Y 113/12005

(57) ABSTRACT

The present disclosure provides a degron comprising an amino acid sequence having at least 70% sequence identity to an amino acid sequence as set forth in SEQ ID NO: 1 for regulating the expression of a protein. The present disclosure also provides a fusion protein comprising a polypeptide of interest and the degron. Further, the present disclosure relates to a method of producing the fusion protein and a method of regulating the expression of a polypeptide of interest.

### SEQUENCE LISTING

This application contains a Sequence Listing which has been submitted electronically as an XML file. The Sequence Listing file is entitled PD054271IN-SC_SEQUENCELISTING_XML, is 60 kilobytes in size, and has a creation date of Feb. 19, 2024. The information in the electronic format of the Sequence Listing is incorporated herein by reference in its entirety.

### FIELD OF INVENTION

The present disclosure broadly relates to the field of molecular biology and genetic engineering. The present disclosure in particular relates to a degron and a method of regulating protein expression using said degron.

### BACKGROUND OF THE INVENTION

In cells, proteins are expressed in a precise quantity. Any deviation in the protein expression from the precise quantity can result in loss of function or aggregation or unwanted side-effects. Studying such deviations in protein expression is essential as well as challenging. Similarly, understanding the physiological function of essential genes in cell is very difficult as they cannot be deleted or knocked out. A system that can achieve partial expression of such genes will be valuable to investigate the physiological function of essential genes.

Similar to endogenous proteins as described herein, there is a need for achieving a graded expression of exogenous proteins, to aid the in vitro investigations in molecular biology. The expression of exogenous proteins in mammalian cells depends on several factors including the vector used, the promoter controlling the expression, sequence of the protein to be expressed, cell type in which the protein is to be expressed, the stability of the mRNA encoding the protein and the stability of the protein itself.

Therefore, there is a need for a robust technique to regulate the expression of an exogenous or an endogenous protein.

### SUMMARY OF INVENTION

In a first aspect of the present disclosure, there is provided a degron comprising an amino acid sequence having at least 70% sequence identity to an amino acid sequence as set forth in SEQ ID NO: 1 for regulating the expression of a protein.

In a second aspect of the present disclosure, there is provided a fusion protein, comprising: a) a polypeptide of interest; and b) the degron as disclosed herein; wherein the degron is operably linked to the polypeptide of interest; and the fusion protein has an amino acid sequence selected from a group consisting of SEQ ID NO: 17, SEQ ID NO: 18, SEQ ID NO: 19 and SEQ ID NO: 20.

In a third aspect of the present disclosure, there is provided a polynucleotide encoding the fusion protein as disclosed herein.

In a fourth aspect of the present disclosure, there is provided a nucleotide construct comprising the polynucleotide as disclosed herein, operably linked to a promoter; wherein the promoter is selected from a group consisting of CMV promoter, EF1α promoter, chicken β-actin promoter, SV40 promoter, Ubc promoter, and CAG promoter.

In a fifth aspect of the present disclosure, there is provided an expression vector comprising the nucleotide construct as disclosed herein.

In a sixth aspect of the present disclosure, there is provided a host cell comprising the nucleotide construct as disclosed herein or the expression vector as disclosed herein.

In a seventh aspect of the present disclosure, there is provided a method of producing a fusion protein as disclosed herein, wherein the method comprises: a) transforming a host cell with the nucleotide construct as disclosed herein or the expression vector as disclosed herein to obtain a transformed host cell; and b) culturing the transformed host cell under conditions favouring the production of the fusion protein.

In an eighth aspect of the present disclosure, there is provided a method for regulating the expression of a polypeptide of interest, wherein the method comprises: a) transforming a host cell with the nucleotide construct as disclosed herein or the expression vector as disclosed herein; b) culturing the transformed host cell under conditions favouring the production of the fusion protein; and c) detecting the presence or absence of a change in the expression of the polypeptide of interest in the transformed host cell with reference to a host cell not transformed with said nucleotide construct.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate the aspects of the present disclosure. The disclosure may be better understood by reference to the drawings in combination with the detailed description of the specific embodiments presented herein.

FIG. 1A depicts schematic of the MTCH2xx constructs used in the assay. FIG. 1B depicts Western blot of lysates from HEK293 cells transfected with the different FLAG-HA-tagged MTCH2 isoforms, namely MTCH2, MTCH2x, MTCH2xx with and without the predicted degron. FIG. 1C depicts quantification of amount of MTCH2xx isoforms with and without the degron from three independent experiments is shown as a bar graph. Graph shows Mean±SE, in accordance with the embodiments herein.

FIG. 2A depicts the schematic of a pCDNA construct with a GFP degron with a stop codon and FIG. 2B depicts the schematic of a pCDNA construct with a GFP degron without stop codon, in accordance with the embodiments herein.

FIG. 3A depicts fluorescence microscopy images, and FIG. 3B depicts flow cytometry analyses (Mean±SD, n=3) of HEK293 cells transfected with the GFP constructs. FIG. 3C depicts Western blotting and RT-PCR analyses from lysates of HEK293 cells transfected with constructs encoding GFP with or without the degron, or with a stop codon between GFP and the degron, and lysates of untransfected HEK293 cells as control (UT), in accordance with the embodiments herein.

FIG. 4A depicts fluorescence microscopy, and FIG. 4B depicts Western blotting analyses from lysates of HEK293 cells transfected with the GFP expressing constructs and treated with the proteasomal inhibitor, MG132, in accordance with the embodiments herein.

FIG. 5A depicts flow cytometry, and FIG. 5B depicts Western blotting analyses of lysates from HEK293 cells transfected with the GFP expressing constructs in which either the wild-type degron or mutant degron sequence was present. For the graph in FIG. 5A: Mean±SD, n=3, in accordance with the embodiments herein.

FIG. 6A depicts schematic of construct comprising the degron sequence cloned downstream of and in-frame with the coding sequence of Renilla luciferase such that it is translated along with RLuc. FIG. 6B depicts luminescence assay readings (Mean±SE, n=3) from HEK293 cells transfected with the RLuc constructs. FIG. 6C depicts RT-PCR analyses from lysates of HEK293 cells transfected with the RLuc constructs, along with lysates of untransfected HEK293 cells as control (UT), in accordance with the embodiments herein.

FIG. 7 depicts the fluorescence microscopy images of HEK293 cells transfected with mtDsRed (mitochondrially targeted DsRed)-degron with and without a stop codon, in accordance with the embodiments herein.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

As discussed in the background, there is a need for a technique to obtain graded expression of endogenous or exogenous proteins. Accordingly, the present disclosure provides a technique to regulate (fine-tune) the expression of an exogenous protein by addition of different small peptide tags, called degrons, which provides an additional level of control over protein expression. Particularly, the present disclosure provides a short putative degron near the C-terminus of MTCH2xx protein. Further, the present disclosure describes that the short nine amino acid peptide can be fused to an exogenous protein to bring about a significant reduction in protein expression. Also, mutations and deletions have been carried out in this peptide sequence to obtain short tags that result in a variety of expression levels of a polypeptide of interest.

### Degron

Embodiments herein include a degron.

The term “degron”, as used herein refers to short peptide sequences that regulate the protein expression by directing them towards degradation.

In an embodiment of the present disclosure, there is provided a degron comprising an amino acid sequence having at least 70% sequence identity to an amino acid sequence as set forth in SEQ ID NO: 1 for regulating the expression of a protein. In another embodiment of the present disclosure, there is provided a degron comprising an amino acid sequence having at least 70%, at least 75%, at least 80%, at least 85%, at least 90%, or at least 95% sequence identity to an amino acid sequence as set forth in SEQ ID NO: 1 for regulating the expression of a protein. In yet another embodiment of the present disclosure, there is provided a degron comprising an amino acid sequence as set forth in SEQ ID NO: 1 for regulating the expression of a protein.

The degron sequence may be fused at the N terminus or C terminus of a protein whose expression is to be regulated. According to the present disclosure, the degron sequence is fused at the C-terminus or in proximity to the C-terminus of a protein. In an embodiment of the present disclosure, the degron is fused at the C-terminus of the protein.

In some embodiments, the wild type degron sequence may be modified to obtain mutants that still retain the ability to regulate the protein expression by directing them towards degradation. Mutants or analogs may be prepared by the deletion of a portion of the sequence encoding the degron, by insertion of a sequence, and/or by substitution of one or more nucleotides within the sequence. Techniques for modifying nucleotide sequences, such as site-directed mutagenesis, are well known to those skilled in the art. The mutants of degron that may be generated is not limited to the mutants disclosed in the present disclosure.

In an embodiment of the present disclosure, the degron comprises an amino acid sequence selected from the group consisting of SEQ ID NO: 2, SEQ ID NO: 3, SEQ ID NO: 4, SEQ ID NO: 5, SEQ ID NO: 6, SEQ ID NO: 7, and SEQ ID NO: 8; wherein SEQ ID NO: 2 to SEQ ID NO: 8 are mutant sequences of the wild type degron sequence.

In an embodiment of the present disclosure, there is provided a degron comprising an amino acid sequence having at least 70%, at least 75%, at least 80%, at least 85%, at least 90%, or at least 95% sequence identity to an amino acid sequence as set forth in SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8 for regulating the expression of a protein.

In an embodiment of the present disclosure, the degron is encoded by a nucleotide sequence, having a sequence selected from the group consisting of SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16.

### Fusion Protein

The present disclosure also provides a fusion protein comprising the degron disclosed in various embodiments herein.

The term “fusion protein,” or “degron fusion protein,” as used herein refers to a fusion comprising a degron and a selected polypeptide of interest as part of a single continuous chain of amino acids, which does not occur in nature.

In an embodiment of the present disclosure, there is provided a fusion protein, comprising a polypeptide of interest; and the degron as disclosed herein; wherein the degron is operably linked to the polypeptide of interest; and the fusion protein has an amino acid sequence selected from a group consisting of SEQ ID NO: 17, SEQ ID NO: 18, SEQ ID NO: 19, and SEQ ID NO: 20.

The term “polypeptide of interest” as used herein refers to a polypeptide that is intended for fusion with a degron to achieve a graded expression of the polypeptide of interest. The polypeptide of interest may be a membrane protein, a receptor, a hormone, a transport protein, a transcription factor, a cytoskeletal protein, an extracellular matrix protein, a signal-transduction protein, an enzyme, or any other protein of interest. The polypeptide of interest may comprise an entire protein, or a biologically active domain (e.g., a catalytic domain, a ligand binding domain, or a protein-protein interaction domain), or a polypeptide fragment of a selected protein.

In an embodiment of the present disclosure, the polypeptide of interest is selected from the group consisting of GFP, MTCH, and Renilla luciferase.

The fusion proteins may also contain sequences exogenous to the degron, and polypeptide of interest. For example, the fusion may include targeting or localization sequences, detectable labels, or tag sequences.

In an embodiment of the present disclosure, the fusion protein further comprises a tag and/or a detectable label.

The term “tag” as used herein refers to a molecule used for facilitating the monitoring of the production and degradation of the fusion protein. Further, the tag may also be used for the downstream processing of the fusion protein. Examples of tag include but are not limited to FLAG, HA, myc, His, GST, V5, MBP, or Biotin.

In some embodiments the tag may be present at the N-terminus or C-terminus of the fusion protein. Preferably, the tag is present at the C-terminus.

The term “detectable label” as used herein refers to a molecule capable of detection and enables the detection of other substances/molecules that are in association with the detectable label. The detectable label may be a fluorescent label or a luminescent label. Examples of fluorescent labels include but are not limited to green fluorescent protein (GFP), mCherry, red fluorescent protein (RFP), DsRed (variant of RFP), yellow fluorescent protein (YFP), cyan fluorescent protein (CFP). Examples of luminescent labels include but are not limited to Firefly luciferase, Renilla luciferase, nanoLuc, or Gaussia luciferase.

In an embodiment of the present disclosure, the tag is selected from FLAG, HA, myc, His, GST, V5, MBP, or Biotin; and the detectable label is selected from fluorescent label (GFP, mCherry, RFP, DsRed, YFP, CFP), or luminescent label (Firefly luciferase, Renilla luciferase, nanoLuc, Gaussia luciferase).

In some embodiments, the fusion protein may further comprise a targeting sequence, which facilitates the localization of the protein at specific target sites in the cell, such as the mitochondria, endoplasmic reticulum, nucleus, or nucleolus.

### Polynucleotide

The present disclosure also provides a polynucleotide encoding the fusion protein disclosed in various embodiments herein.

In an embodiment of the present disclosure, there is provided a polynucleotide encoding the fusion protein as disclosed herein.

In an embodiment of the present disclosure, the polynucleotide has a nucleotide sequence selected from the group consisting of SEQ ID NO: 21, SEQ ID NO: 22, SEQ ID NO: 23, and SEQ ID NO: 24.

### Nucleotide Construct

The present disclosure also provides a nucleotide construct comprising the polynucleotides disclosed in various embodiments herein.

In an embodiment of the present disclosure, the nucleotide construct comprises the polynucleotide as disclosed herein, operably linked to a promoter.

The promoter may be an inducible promoter or constitutive promoter. Examples of promoters that may be used in the present disclosure include CMV (cytomegalovirus) promoter, EF1α promoter, chicken β-actin promoter, SV40 promoter, Ubc promoter, and CAG (CMV early enhancer/chicken β actin) promoter.

In an embodiment of the present disclosure, the nucleotide construct comprises the polynucleotide as disclosed herein, operably linked to a promoter, wherein the promoter is selected from a group consisting of CMV promoter, EF1α promoter, chicken β-actin promoter, SV40 promoter, Ubc promoter, and CAG promoter.

In an embodiment of the present disclosure, the nucleotide construct has a nucleotide sequence selected from SEQ ID NO: 25, SEQ ID NO: 26, SEQ ID NO: 27 and SEQ ID NO: 28.

### Expression Vector

The present disclosure further provides an expression vector comprising the nucleotide construct disclosed in various embodiments herein.

In an embodiment of the present disclosure, there is a provided an expression vector comprising the nucleotide vector as disclosed herein.

Various vectors are generally known and may be used in achieving the expression vector according to embodiments herein. Examples include, but are not limited to mammalian expression vectors, such as adenoviruses, and lentiviruses;

and plasmids including pcDNA, pIRESneo, pLVX, pIRESpuro, pBApo-CMV Pur DNA, pBI-CMV1, and pEF1alpha-IRES.

### Host Cell

Further, the present disclosure provides recombinant host cells transfected with the expression vector or the nucleotide construct comprising the polynucleotide encoding the fusion protein disclosed in various embodiments herein. In an embodiment, the host cell is a eukaryotic cell selected from a group consisting of HEK293, HeLa, K562, Neuro2a, U2OS, HepG2, A542, CACO2, CaOV3, Saccharomyces cerevisiae, and Saccharomyces pombe.

### Methods and Applications

The present disclosure provides a method of producing the fusion protein disclosed in various embodiments herein.

In an embodiment of the present disclosure, the method of producing a fusion protein comprises a) transforming a host cell with the nucleotide construct as disclosed herein or the expression vector disclosed herein to obtain a transformed host cell; and b) culturing the transformed host cell under conditions favouring the production of the fusion protein. Various techniques for preparing polynucleotides, transfection, and culturing are known in the art which may be used in various embodiments.

The present disclosure provides a method for regulating the expression of a polypeptide of interest. In an embodiment of the present disclosure, the method for regulating the expression of a polypeptide of interest comprises: a) transforming a host cell with the nucleotide construct as disclosed herein; or the expression vector as disclosed herein; b) culturing the transformed host cell under conditions favoring the production of the fusion protein; and c) detecting the presence or absence of a change in the expression of the polypeptide of interest in the transformed host cell with reference to a host cell not transformed with said nucleotide construct or said expression vector.

### Sequences Used in the Present Disclosure

| Sequence Description | SEQ ID NO: |
|---|---|
| Amino acid sequence of Degron Wildtype | SEQ ID NO 1 |
| Amino acid sequence of Degron No F | SEQ ID NO 2 |
| Amino acid sequence of Degron No L | SEQ ID NO 3 |
| Amino acid sequence of Degron W-A | SEQ ID NO 4 |
| Amino acid sequence of Degron No F No L | SEQ ID NO 5 |
| Amino acid sequence of Degron No F W-A | SEQ ID NO 6 |
| Amino acid sequence of Degron No L W-A | SEQ ID NO 7 |
| Amino acid sequence of Degron No F No L W-A | SEQ ID NO 8 |
| Nucleotide sequence of Degron Wildtype | SEQ ID NO 9 |
| Nucleotide sequence of Degron No F | SEQ ID NO 10 |
| Nucleotide sequence of Degron No L | SEQ ID NO 11 |
| Nucleotide sequence of Degron W-A | SEQ ID NO 12 |
| Nucleotide sequence of Degron No F No L | SEQ ID NO 13 |
| Nucleotide sequence of Degron No F W-A | SEQ ID NO 14 |
| Nucleotide sequence of Degron No L W-A | SEQ ID NO 15 |
| Nucleotide sequence of Degron No F No L W-A | SEQ ID NO 16 |

DEGRON FUSION PROTEINS

| | |
|---|---|
| Amino acid sequence of Fusion protein with GFP | SEQ ID NO 17 |
| Amino acid sequence of Fusion protein with | SEQ ID NO 18 |
| MTCH | |
| Amino acid sequence of Fusion protein with | SEQ ID NO 19 |
| Renilla luciferase | |
| Amino acid sequence of Fusion protein with | SEQ ID NO 20 |
| mtDsRed | |
| Nucleotide sequence of Fusion protein with GFP | SEQ ID NO. 21 |
| Nucleotide sequence of Fusion protein with | SEQ ID NO. 22 |
| MTCH | |
| Nucleotide sequence of Fusion protein with | SEQ ID NO. 23 |
| Renilla luciferase | |
| Nucleotide sequence of Fusion protein with | SEQ ID NO. 24 |
| mtDsRed | |

FUSION PROTEIN CONSTRUCTS

| | |
|---|---|
| Nucleotide Sequence of The Construct Coding for | SEQ ID NO: 25 |
| GFP Degron Fusion Protein | |
| Nucleotide Sequence of The Construct Coding for | SEQ ID NO: 26 |
| MTCH Degron Fusion Protein | |
| Nucleotide Sequence of The Construct Coding for | SEQ ID NO: 27 |
| Renilla Degron Fusion Protein | |
| Nucleotide Sequence of The Construct Coding for | SEQ ID NO: 28 |
| mtDsRed Degron Fusion Protein | |

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

### Materials

The procurement details of the reagents and chemicals used in the present disclosure is as follows.

Lipofectamine 2000, used for transfection in HEK293 cells was procured from Thermo Fisher Scientific. Anti-GFP antibody (BioLegend), anti-HA antibody (SIGMA), anti-Actin antibody (SIGMA) and horseradish peroxidase-conjugated secondary antibodies (Invitrogen) were used as per the manufacturer's instructions. Protein concentration was measured using BioRad Protein Assay Dye Reagent. The development of blots was carried out using Clarity ECL (Bio-Rad) in ChemiDoc Imaging System (Bio-Rad) or LAS-4000 imager (Fujifilm). MG132, a proteasomal inhibitor was procured from SIGMA and Abcam. For luminescence-based assays, the Dual Luciferase Reporter Assay System from Promega was used and the measurement was carried out using the Glomax Explorer (Promega). Fluorescence was analyzed using fluorescence microscopy (Olympus IX73) and by flow cytometry (CytoFLEX S, Beckman Coulter). Enzymes for PCR amplification and cloning were purchased from NEB and Thermo Fisher Scientific.

### Example 1

### Identification of Degron Sequence

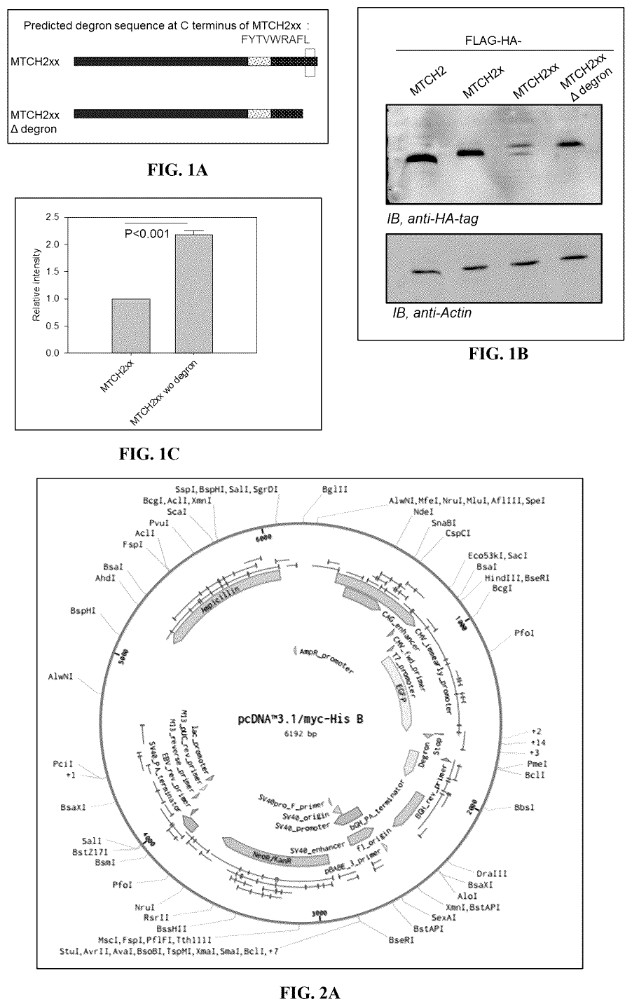

In an earlier study it was shown that MTCH2 mRNA undergoes canonical translation, as well as single and double-stop codon readthrough (SCR). MTCH2xx, a double-SCR isoform arising from MTCH2 mRNA was found to have reduced half-life due to proteasomal degradation (Manjunath, L. E., Singh, A., Sahoo, S., Mishra, A., Padmarajan, J., Basavaraju, C. G., & Eswarappa, S. M. (2020). Stop codon read-through of mammalian MTCH2 leading to an unstable isoform regulates mitochondrial membrane potential. Journal Title Journal of Biological Chemistry, 295 (50), 17009-17026). Bioinformatic analysis using Eukaryotic Linear Motif (ELM) predictor (Kumar, M., Michael, S., Alvarado-Valverde, J., McRossed D Sign©száros, B., Sámano-Sánchez, H., Zeke, A., Dobson, L., Lazar, T., Örd, M., Nagpal, A., Farahi, N., Käser, M., Kraleti, R., Davey, N. E., Pancsa, R., Chemes, L. B., & Gibson, T. J. (2022). The Eukaryotic Linear Motif resource: 2022 release. Nucleic Acids Research, 50 (DI), D497-D508) of the C-terminus region unique to the MTCH2xx isoform, showed the presence of a putative degron—FYTVWRAFL—(SEQ ID NO: 1) present in the C-terminus of the double-SCR product, MTCH2xx. To confirm the functional role of the putative degron sequence, MTCH2xx was cloned with/without the C-terminus containing the predicted degron sequence (Schematic in FIG. 1A). HEK293 cells were transfected with plasmids expressing FLAG-HA-tagged MTCH2 isoforms. Among the MTCH2 isoforms, MTCH2 is the canonical isoform; MTCH2x and MTCH2xx are products of single and double SCR, respectively. MTCH2xx is longer than MTCH2x, which is in turn longer than MTCH2. Western blotting was performed to determine the amount of MTCH2xx isoforms with and without the degron from three independent experiments. Two-tailed Student's t-test was used to calculate the P value.

### Results

Western blot analysis from HEK293 cells transfected with FLAG-HA-tagged-MTCH2 constructs showed that the absence of the predicted degron increased the expression levels of MTCH2xx (FIG. 1B and quantification in FIG. 1C).

### Example 2

### Effect of Degron on the Expression of Green Fluorescent Protein (GFP)

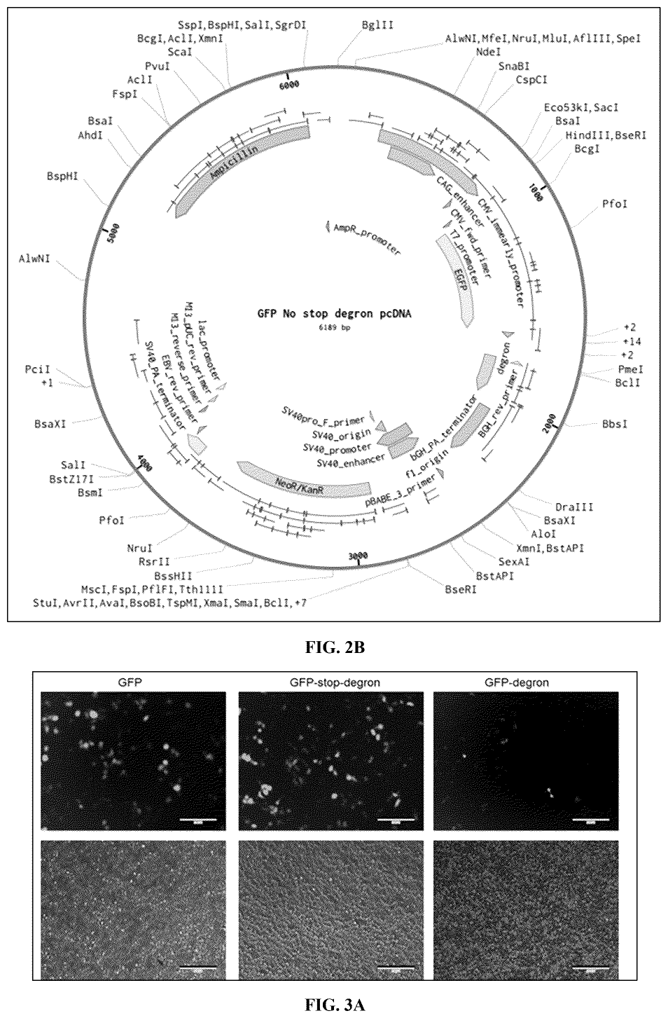

The degron comprising of 9 amino acids (SEQ ID NO. 1) was cloned downstream of the coding sequence of green fluorescent protein (GFP) (FIG. 2B). A construct in which a stop codon separated GFP from the degron served as a control (FIG. 2A) to test if the reduced expression was because of the RNA sequence or the translated peptide. The constructs were transfected in HEK293 cells. Flow cytometry, Western blotting and RT-PCR analyses were performed using the lysates of HEK293 cells transfected with constructs encoding GFP with (SEQ ID NO: 25) or without the degron, or with a stop codon between GFP and the degron. Two-tailed Student's t test was used to calculate the p value, to determine statistical significance.

Further, to determine if any reduced expression observed after translation of the degron was because of proteasomal degradation, HEK293 cells were transfected with the GFP-expressing constructs and the cells were treated with 10 μM of the proteasomal inhibitor, MG132, for 5 h. Fluorescence microscopy and Western blotting were carried out with the lysates.

Fluorescence-based assay: HEK293 cells were seeded at 70-80% confluency in a 24 well plate. The cells were transfected with 500 ng/well of the fluorescence-encoding reporter constructs using Lipofectamine 2000, following manufacturer's instructions. 24 hours post-transfection, the samples were subjected to fluorescence microscopy (Olympus IX73) and flow cytometry (CytoFLEX S).

Luminescence-based assay: HEK293 cells were seeded at 75-95% confluence in 24 well plates. 250 ng/well of Renilla luciferase-encoding reporters along with firefly luciferase-encoding plasmid as transfection control were co-transfected using Lipofectamine 2000, following manufacturer's instructions. 24 hours post-transfection, the FLuc and RLuc activities were measured using Dual Luciferase Reporter Assay System (Promega) in the Glomax Explorer (Promega).

Western blotting: HEK293 cells transfected with the mentioned constructs were lysed using cell lysis buffer (20 mM Tris-HCl, 150 mM NaCl, 1 mM EDTA, 1% Triton X-100 with protease inhibitor cocktail (Promega)). The concentration of the proteins was measured using Protein Assay Dye Reagent (Bio-Rad). 20-50 ug of the cell lysate was subjected to denaturing SDS-PAGE in a 12% Tris-Glycine gel. The proteins were transferred to a PVDF membrane (Merck) and blocked with a blocking agent (5% skimmed milk in PBS). The membrane was then probed with the primary antibody overnight and followed by probing with the corresponding secondary antibody. The blot was developed using Clarity ECL reagent (Bio-Rad) and the images were recorded using LAS-4000 imager (Fujifilm) or ChemiDoc Imaging System (Bio-Rad).

RT-PCR: Total RNA from cells was isolated using RNAiso Plus (TaKaRa). cDNA synthesis was carried out with 500 ng of RNA using olgi (dT) reverse primer and RevertAid Reverse Transcriptase (Thermo Fisher). Semi-quantitative analysis of the mRNA was carried out using gene-specific primers.

### Results

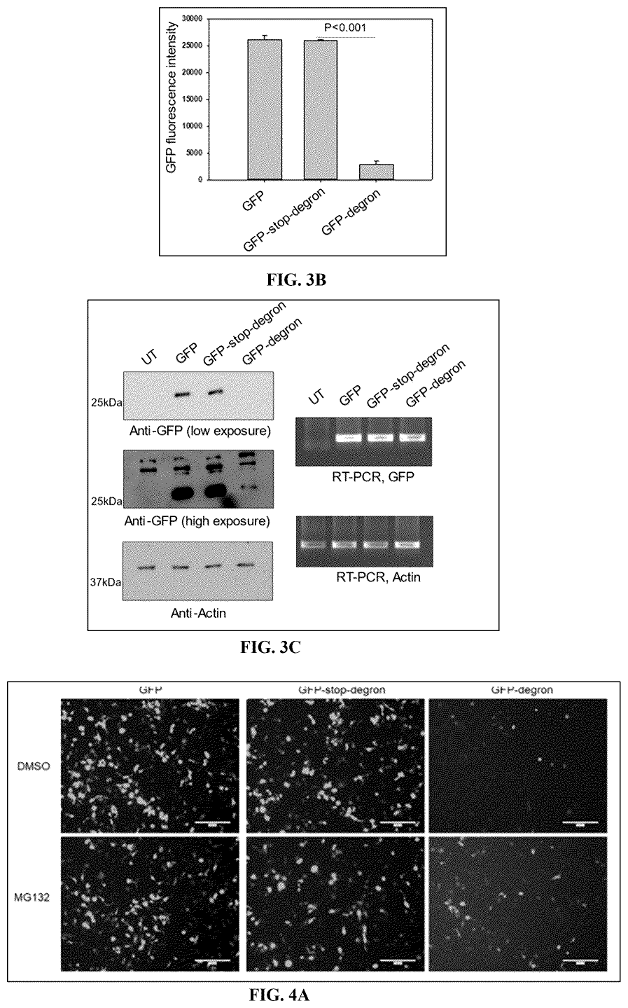

Fluorescence microscopy images showed reduced fluorescence in the construct without a stop codon between GFP and the degron (FIG. 3A). The same result was confirmed by flow cytometry (FIG. 3B) and by Western blotting (FIG. 3C). RT-PCR analysis showed that there was no difference in the RNA levels of these constructs (FIG. 3D).

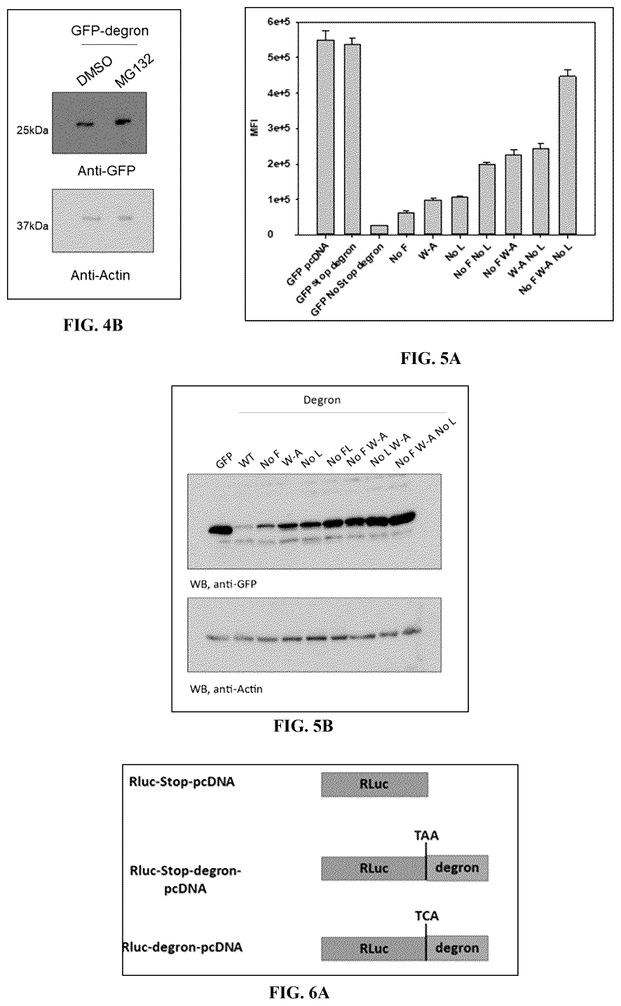

Partial increase in expression of GFP-degron was seen in the presence of MG132 by both fluorescence microscopy (FIG. 4A) and Western blotting (FIG. 4B), which confirmed that the degron targets the protein generated towards proteasomal degradation.

### Example 3

### Effect of Degron Mutants in Regulating the Expression of GFP

To check the effect of deleting or mutating different amino acids in the degron on the levels of the GFP fusion protein (SEQ ID NO:17) several degron mutants were generated. (F), Leucin (L) amino acids were deleted, and Tryptophan was mutated to alanine (W-A), the three residues predicted to be important for this category of degrons (Kussie, P. H., Gorina, S., Marechal, V., Elenbaas, B., Moreau, J., Levine, A. J., & Pavletich, N. P. (1996). Structure of the MDM2 oncoprotein bound to the p53 tumor suppressor transactivation domain. Science (New York, N.Y.), 274 (5289), 948-953). Also, additional degron mutants were generated by combinations (double or triple) of mutations or deletions of these three residues (as shown in Table 1). Flow cytometry and Western blotting analysis was carried out with the lysates from HEK293 cells transfected with the GFP expressing constructs in which either the wild-type degron or mutant degron sequence was present, for three independent experiments. Two-tailed Student's t test was used to calculate the p value, to determine statistical significance.

TABLE 1

| | SEQ ID | | | SEQ |
|---|---|---|---|---|
| | of amino | | | ID of |
| | acid | | | nucleotide |
| | sequence | Degron/ | | sequence |
| | of | mutant | | of |
| Degron | Degron/ | amino acid | Degron/mutant | Degron/ |
| description | mutant | sequence | nucleotide sequence | mutants |
| Degron | SEQ ID | FYTVWRAFL | TTCTATACAGTGTGGCGCGCTT | SEQ |
| Wildtype | NO: 1 | | TTTTA | ID NO: |
| | | | | 9 |
| Degron | SEQ ID | YTVWRAFL | TATACAGTGTGGCGCGCTTTTT | SEQ |
| No F | NO: 2 | | TA | ID NO: |
| | | | | 10 |
| Degron | SEQ ID | FYTVWRAF | TTCTATACAGTGTGGCGCGCTT | SEQ |
| No L | NO: 3 | | TT | ID NO: |
| | | | | 11 |
| Degron | SEQ ID | FYTVARAFL | TTCTATACAGTGGCGCGCGCTT | SEQ |
| W-A | NO: 4 | | TTTTA | ID NO: |
| | | | | 12 |
| Degron | SEQ ID | YTVWRAF | TATACAGTGTGGCGCGCTTTT | SEQ |
| No F No L | NO: 5 | | | ID NO: |
| | | | | 13 |
| Degron | SEQ ID | YTVARAFL | TATACAGTGGCGCGCGCTTTTT | SEQ |
| No FW-A | NO: 6 | | TA | ID NO: |
| | | | | 14 |
| Degron | SEQ ID | FYTVARAF | TTCTATACAGTGGCGCGCGCTT | SEQ |
| No L W-A | NO: 7 | | TT | ID NO: |
| | | | | 15 |
| Degron | SEQ ID | YTVARAF | TATACAGTGGCGCGCGCTTTT | SEQ |
| No F No L | NO: 8 | | | ID NO: |
| W-A | | | | 16 |

### Results

Flow cytometry (FIG. 5A) and Western blotting (FIG. 5B) results showed that each mutant resulted in a fusion protein with expression different from either the construct lacking the degron or the construct with the wild-type degron sequence. This enabled a range of expression levels of GFP.

### Example 4

Effect of Degron on the Expression of Renilla Luciferase (RLuc) and Mitochondrially Targeted Fluorescent Protein (mtDsRed)

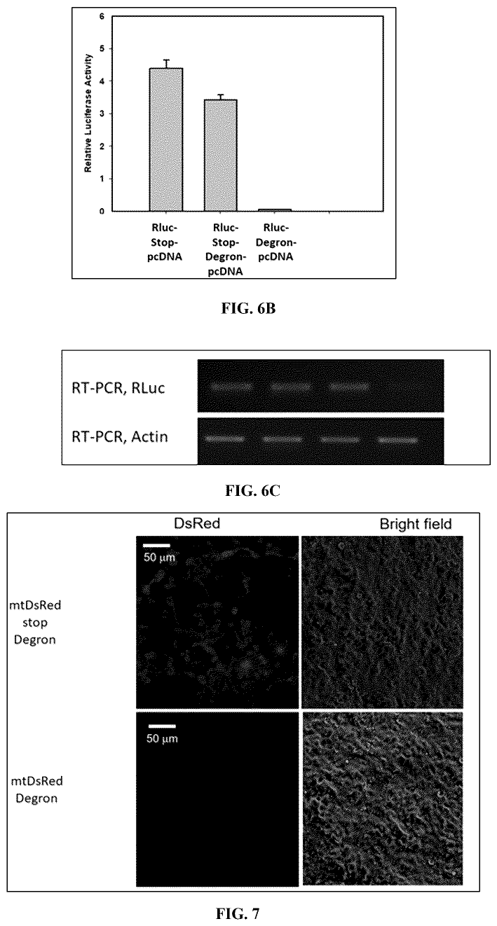

The degron sequence was cloned downstream of and in-frame with the coding sequence of Renilla luciferase (Construct-SEQ ID NO. 27) such that it was translated along with RLuc (FIG. 6A). RT-PCR analysis and luminescence assay were carried out with lysates from HEK293 cells transfected with the RLuc constructs. Two-tailed Student's t test was used to calculate the p value to determine statistical significance of three independent experiments.

Similarly, the effect of degron on the expression of a mitochondrially targeted fluorescent protein was studied by cloning the degron sequence down stream of mtDsRed (Construct-SEQ ID NO. 28). Fluorescence microscopy images of HEK293 cells transfected with mtDsRed-degron with and without a stop codon were obtained.

### Results

The degron when fused to Renilla luciferase reduced the expression of the luciferase-fusion protein (SEQ ID NO: 19) as shown in the luminescence assay result (FIG. 6B) and RT PCR analysis (FIG. 6C).

Similarly, the degron when fused to mitochondrially targeted DsRed fluorescent protein (SEQ ID NO: 20) reduced the expression of the mtDsRed protein (FIG. 7).

Overall, the present disclosure provides a degron and its mutants, which can be used to regulate the expression of a protein of interest at various levels.

### Advantages of the Present Disclosure

The present disclosure provides the degron (FYTVWRAFL) and its mutants (single, double, or triple), which has been used to obtain different levels of expression of a target protein by the fusion of different short peptides of the degron.

The identified degron is present at the C-terminus of the MTCH2xx protein. The fusion of this degron, comprising of 9 amino acids, to the fluorescent reporter, GFP, drastically reduced the expression of GFP.

The disclosed method can be used to optimize expression of any protein in mammalian cells, which has commercial value in industry using mammalian protein expression systems.

The disclosed method can also be used in basic science laboratories interested in studying the function of a given protein.

The disclosed method can be used to investigate the function of essential genes, which is achieved by appending the degron of the present disclosure downstream of the gene of interest such that it is expressed at very low level just enough to maintain the viability of the cells. Then the cell phenotype due to very low expression of that gene is investigated.

The disclosed mutations and deletions in the degron were done to get an array of short tags, which when fused to GFP, resulted in differing levels of reporter expression. This when extended to the expression of other exogenous proteins, such as RLuc, mtDsRed allowed control over the levels of the exogenous protein, by fusion with a short C-terminal tag.

The disclosed degron can also be used to tag protein coding regions at the genome level to regulate the expression levels of endogenous proteins as well. This allows for control of expression at the protein level as opposed to the use of different promoters for differential expression.

I/We claim:

1. A degron comprising an amino acid sequence having at least 70% sequence identity to an amino acid sequence as set forth in SEQ ID NO: 1 for regulating the expression of a protein.

2. The degron as claimed in claim 1, wherein the degron comprises an amino acid sequence selected from the group consisting of SEQ ID NO: 2, SEQ ID NO: 3, SEQ ID NO: 4, SEQ ID NO: 5, SEQ ID NO: 6, SEQ ID NO: 7, and SEQ ID NO: 8.

3. The degron as claimed in claim 1, wherein the degron is encoded by a nucleotide sequence, having a sequence selected from the group consisting of SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16.

4. The degron as claimed in claim 1, wherein the degron is present at the C-terminus of the protein.

5. A fusion protein, comprising:
a) a polypeptide of interest; and
b) the degron as claimed in claim 1;
wherein the degron is operably linked to the polypeptide of interest;
and the fusion protein has an amino acid sequence selected from a group consisting of SEQ ID NO: 17, SEQ ID NO: 18, SEQ ID NO: 19, and SEQ ID NO: 20.

6. The fusion protein as claimed in claim 5, wherein the fusion protein further comprises a tag and/or a detectable label.

7. The fusion protein as claimed in claim 6, wherein the tag is selected from FLAG, HA, myc, His, GST, V5, MBP, or Biotin; and the detectable label is selected from fluorescent label (GFP, mCherry, RFP, DsRed, YFP, CFP), or luminescent label (Firefly luciferase, Renilla luciferase, nanoLuc, Gaussia luciferase).

8. The fusion protein as claimed in claim 5, wherein the polypeptide of interest is selected from the group consisting of GFP, MTCH, Renilla luciferase, and mtDsRed.

9. A polynucleotide encoding the fusion protein as claimed in claim 5.

10. The polynucleotide as claimed in claim 9, wherein the polynucleotide has a nucleotide sequence selected from the group consisting of SEQ ID NO: 21, SEQ ID MO: 22, SEQ ID NO: 23, and SEQ ID NO: 24.

11. A nucleotide construct comprising the polynucleotide as claimed in claim 9, operably linked to a promoter; wherein the promoter is selected from a group consisting of CMV promoter, EF1α promoter, chicken β-actin promoter, SV40 promoter, Ubc promoter, and CAG promoter.

12. The nucleotide construct as claimed in claim 11, wherein the nucleotide construct has a nucleotide sequence selected from SEQ ID NO: 25, SEQ ID NO: 26, SEQ ID NO: 27, or SEQ ID NO: 28.

13. An expression vector comprising the nucleotide construct as claimed in claim 11.

14. A host cell comprising the nucleotide construct as claimed in claim 11.

15. The host cell as claimed in claim 14, wherein the host cell is a eukaryotic cell; and the eukaryotic cell is selected from a group consisting of HEK293, HeLa, K562, Neuro2a, U2OS, HepG2, A542, CACO2, CaOV3, Saccharomyces cerevisiae, and Saccharomyces pombe.

16. A host cell comprising the expression vector as claimed in claim 13.

17. The host cell as claimed in claim 15, wherein the host cell is a eukaryotic cell; and the eukaryotic cell is selected from a group consisting of HEK293, HeLa, K562, Neuro2a, U2OS, HepG2, A542, CACO2, CaOV3, Saccharomyces cerevisiae, and Saccharomyces pombe.

18. A method of producing a fusion protein comprising a polypeptide of interest and a degron comprising an amino acid sequence having at least 70% sequence identity to an amino acid sequence as set forth in SEQ ID NO: 1 for regulating the expression of a protein wherein the degron is operably linked to the polypeptide of interest; and the fusion protein has an amino acid sequence selected from a group consisting of SEQ ID NO: 17, SEQ ID NO: 18, SEQ ID NO: 19, and SEQ ID NO: 20, wherein the method comprises:
a) transforming a host cell with the nucleotide construct as claimed in claim 11 to obtain a transformed host cell; and
b) culturing the transformed host cell under conditions favoring the production of the fusion protein.

19. A method of producing a fusion protein comprising a polypeptide of interest and a degron comprising an amino acid sequence having at least 70% sequence identity to an amino acid sequence as set forth in SEQ ID NO: 1 for regulating the expression of a protein wherein the degron is operably linked to the polypeptide of interest; and the fusion protein has an amino acid sequence selected from a group consisting of SEQ ID NO: 17, SEQ ID NO: 18, SEQ ID NO: 19, and SEQ ID NO: 20, wherein the method comprises:
c) transforming a host cell with the expression vector as claimed in claim 13 to obtain a transformed host cell; and
d) culturing the transformed host cell under conditions favoring the production of the fusion protein.

20. A method for regulating the expression of a polypeptide of interest, wherein the method comprises:
a) transforming a host cell with the expression vector as claimed in claim 13;
b) culturing the transformed host cell under conditions favoring the production of the fusion protein; and
c) detecting the presence or absence of a change in the expression of the polypeptide of interest in the transformed host cell with reference to a host cell not transformed with said nucleotide construct.
